(19) United States
(12) Patent Application Publication
(10) Pub. No.: US 2023/0210789 A1
(43) Pub. Date: Jul. 6, 2023

(54) METHODS FOR TREATING MAJOR DEPRESSIVE DISORDER AND TREATMENT-RESISTANT DEPRESSION

(71) Applicants: iX Biopharma Limited, Willetton, Western Australia (AU); iX Biopharma Ltd, Singapore (SG)

(72) Inventors: Yip Hang Eddy LEE, Singapore (SG); Chin Beng Stephen LIM, Willetton, Western Australia (AU)

(21) Appl. No.: 18/000,497

(22) PCT Filed: May 28, 2021
(86) PCT No.: PCT/AU2021/050519

§ 371 Date: Dec. 1, 2022

(30) Foreign Application Priority Data
Jun. 2, 2020 (AU) 2020901810

(51) Int. Cl. A61K 31/135; A61K 47/36; A61K 9/00

(52) U.S. Cl. CPC A61K 31/135; A61K 47/36; A61K 9/006

(57) ABSTRACT

The invention concerns methods of using a ketamine dosage form in treating depression and, in particular, major depressive disorder and treatment-resistant depression, comprising administering to a patient in need thereof, a fast dissolving freeze-dried wafer solid dosage form with a matrix for rapid release and absorption of ketamine in the oral cavity of the said patient.

### FIELD OF THE INVENTION

The present invention relates to a method of using a ketamine dosage form in treating depression and, in particular, major depressive disorder and treatment-resistant depression, comprising administering to a patient in need thereof, a fast dissolving freeze-dried wafer solid dosage form with a matrix for rapid release and absorption of ketamine in the oral cavity of the said patient.

### BACKGROUND

### Depression

Major depressive disorder (MDD) and treatment-resistant depression (TRD) are devastating mental disorders affecting approximately 16 percent of the world population, causing serious health and socio-economic consequences. Although interventions such as pharmacotherapies and cognitive behavioural psychotherapies are available, a high proportion of patients remain treatment-resistant. Moreover, even when effective, existing monoaminergic-based pharmacotherapies often take several weeks or months to exert their full therapeutic effects.

Depression can vary in severity from mild to very severe and can be episodic, recurrent or chronic in nature. Current antidepressant medications augment/potentiate the effects of the neurotransmitters, mainly increasing the concentration of the neurotransmitters in the intrasynaptic area of the neurons.

This mode-of-action takes weeks to months to achieve their full effects. This lag-time to response (non-response period) or “inaction period” allows the patients to continue suffering their depressive symptoms and also the risk of self-harm (suicidal behaviour).

Major depression is related to changes in brain morphology and neural plasticity (hippocampal atrophy) and decreases in neurite outgrowth and neurogenesis. These changes are mediated by altered expression of BDNF (brain-derived neurotrophic factor). Efficacy of antidepressants are related to their ability to increase the expression of BDNF. Increases in BDNF occurs after chronic (10-21 day), but not acute (1 day) during current antidepressant treatment, the possible reason why current antidepressants have a lag period of 10-21 days to response.

Depression and suicidal behavior have recently been shown to be associated with disturbances in structural and synaptic plasticity. The expression of BDNF is decreased in depressed patients.

### Ketamine

Ketamine is a no barbiturate, rapidly-acting general anaesthetic that was first synthesized in 1964. Ketamine hydrochloride has been approved for clinical use as an injectable formulation in the United States since 1970, under the trade name Ketalar®. Ketamine is a racemic drug with a wide margin of safety and has been studied in over 12,000 operative and diagnostic procedures involving over 10,000 patients from 105 separate studies in which Ketalar® was administered as the sole agent, as induction for other general anaesthetic agents, or to supplement low potency agents.

As a general anaesthetic, ketamine rapidly produces a profound state of dissociative anaesthesia. Spontaneous respiration is maintained, and cardiovascular function is not depressed and indeed may be stimulated. Despite the efficacy and safety of ketamine as a general anaesthetic, its use has been limited due to unpleasant psychological experiences that may occur as patients awake from anaesthesia.

In addition to its use as a general anaesthetic, in recent years there has been increasing interest in the use of ketamine at non-anaesthetic low doses as an adjunct in acute and chronic pain management (Visser 2006, Weinbroum 2011, Bell 2006) and as a rapidly-acting anti-depressant (Zarate 2006).

The principal pharmacological action of ketamine is understood to be antagonism at NMDA receptors. Other actions of ketamine may also include activity at central neurotransmitter targets including dopamine, 5-HT, GABA, opioid and endocannabinoid receptors. Ketamine has activity at ATP-sensitive, voltage-gated Ca++ and K+ channels, Ca++ transport and sensitization pathways and Na+ channels. Additional actions are at nicotinic, purinergic, histamine receptors and actions on inflammatory pathways including leukotrienes.

Ketamine has a single chiral centre and both R and S enantiomers of the racemic drug show activity as NMDA antagonists, although the S enantiomer is approximately 3 times more potent in humans in vivo. There is no evidence of chiral inversion in vivo. Both enantiomers appear to be principally metabolised by demethylation to norketamine (NK). R and S norketamine also show activity as NMDA antagonists, although their potency is approximately 5-8 times less than the parent molecules.

Ketamine is eliminated principally by metabolism with the major pathway being hepatic CYP3A4, with a minor contribution from CYP2B6. Terminal half-life of racemic drug is approximately 3 hours. However, the duration of action as an anaesthetic is approximately 30 minutes, depending on dose, being principally determined by redistribution from highly perfused brain to less well perfused tissues, rather than by elimination.

With five decades of clinical use, ketamine has been shown to be a remarkably safe general anaesthetic. Unlike most other general anaesthetics, ketamine does not depress respiratory function and cardiovascular function is not depressed and may be stimulated. However, patients may experience unpleasant psychological symptoms when emerging from ketamine anaesthesia.

### Wafermine

Wafermine™ is a wafer formulation of racemic ketamine in a rapidly dissolving hydrophilic matrix. Wafermine™ is intended for sublingual administration and is being developed for the treatment of moderate to severe acute pain.

Clinical trials with Wafermine™ have shown it to be well tolerated. The most frequent adverse effects are nausea and CNS symptoms such as dizziness and feelings of unreality, with a frequency and intensity related to dose. The wafers were well tolerated in the oral cavity.

### Ketamine and Depression

Placebo-controlled trials have provided some evidence for antidepressant effects of ketamine. Ketamine's routine clinical use for the treatment of depression is restricted due to its dissociative effects, changes in sensory perception, intravenous route of administration, as well as its abuse liability (Zanos 2018).

The first clinical trial reporting antidepressant actions of ketamine was published in 2000, where ketamine was administered intravenously (40-min infusion) at the sub-anesthetic dose of 0.5 mg/kg. This contrasts with the typical dose of ketamine used in anesthesia of up to 2 mg/kg.

A subsequent double blind randomized clinical trial demonstrated the efficacy of ketamine in treatment-resistant major depressed patients, who failed at least two conventional antidepressant treatments. The antidepressant effects of ketamine manifested within 2 hours post-infusion and 35% of patients maintained response for at least 7 days. Following these initial reports, several other clinical trials demonstrated rapid antidepressant actions of ketamine in treatment refractory patients.

Spravato™, intra-nasal (S)-ketamine, was approved by the U.S. FDA in 2019 in combination with an oral anti-depressant for treatment resistant depression.

There is a need in the art for alternative delivery of ketamine for the treatment of depression to improve compliance and uptake by patients. It is an objective of the invention to overcome one or more problems foreshadowed by the prior art.

### SUMMARY OF THE INVENTION

In one aspect, the invention is a method of treating depression, said method comprising administering to a patient in need thereof, a fast dissolving wafer solid dosage form with a matrix for release of a biologically active material in an oral cavity wherein said dosage form comprises:

(a) a biologically active material;
(b) a matrix forming agent;
wherein the dosage form dissolves in the oral cavity without leaving a residue of said
dosage form in the oral cavity that is detectable by a subject, thereby avoiding the urge for the subject to swallow the dosage form; and
wherein said dosage form disintegrates in the oral cavity in a time of less than 15 seconds and dissolves in the oral cavity in a time of less than 60 seconds.

In one preferred embodiment, the solid dosage form is fast disintegrating.

In one preferred embodiment, the wafer is freeze-dried.

In one preferred embodiment, the biologically active material is absorbed by diffusion. Preferably, the biologically active material is absorbed by diffusion directly into the systemic circulation.

In one preferred embodiment, the solid dosage form is delivered sublingually. Preferably, the method provides ketamine sublingual adsorption.

In another preferred embodiment, the biologically active material is selected from the group consisting of: ketamine, an analog, variant, metabolite and a salt form thereof. Preferably, the ketamine is selected from the group consisting of: racemic ketamine, S-ketamine and R-ketamine, and any metabolites (including norketamine, hydroxy-ketamines, hydroxy-norketamines, 5,6-dehydronorketamine, phenol-ketamines and phenol-norketamines) that have or may have a role in ketamine's antidepressant effects. Preferably, the ketamine is an enantiomeric mixture of (R)-ketamine and (S)-ketamine and not (S)-ketamine alone. Preferably, the ketamine is present in an amorphous (non-crystalline) state. Preferably, the ketamine is in the form of an amorphous solid distributed throughout the dosage form.

In another preferred embodiment, the solid dosage form has a pH selected from the range of: between 3.0 and 8.0, and between 5 and 6.

In another preferred embodiment, the matrix forming agent comprises amylopectin.

Preferably, the matrix forming agent comprises amorphous amylopectin. More preferably, the amylopectin is at a concentration from 2% to 17% weight % by dry weight of the composition of the dosage form. Preferably, the matrix forming agent is greater than 96% water soluble. Preferably, the matrix forming agent is >96% non-ionisable.

In another preferred embodiment, the matrix forming agent comprises a carbohydrate. Preferably, the carbohydrate is a low molecular weight crystalline agent. Preferably, the molecular weight crystalline agent is a sugar or sugar alcohol. In one preferred form, the dosage form comprises a carbohydrate chosen from the list consisting of: mannitol, dextrose, lactose, galactose, sorbitol and trehalose at a concentration selected from the group consisting of 0.01 to 99.99%; 0.1% to 99%; 1% to 90%; 2% to 20%, 3% to 15%; 4% top 10%; from 5% to 80% weight % by dry weight of the composition of the dosage form.

In another preferred embodiment, the dosage form comprises sodium carboxymethyl cellulose (CMC) at a concentration from 0.1 to 15% dry weight of the dosage form.

In another preferred embodiment, the powder x-ray diffraction (XRD) spectrum of the dosage form comprises peaks at 2-theta values at approximately 9.58 degrees, 19.68 degrees, and 20.05 degrees. Preferably, the XRD spectrum does not substantially contain the major peaks from crystalline ketamine or its salts.

In another preferred embodiment, the dosage form is fast disintegrating. Preferably, the dosage form disintegrates in the oral cavity in a time of less than 10 seconds. More preferably, the dosage form disintegrates in the oral cavity in a time of less than 5 seconds. Preferably, the dosage form is robust to allow the patient to dispense and hold the dosage without breaking. More preferably, the dosage form dissolves once placed in the oral cavity in a time period selected from the group consisting of: less than 50 seconds, less than 40 seconds, less than 30 seconds, less than 20 seconds, less than 15 seconds, less than 10 seconds, less than 7.5 seconds, less than 5 seconds, less than 4 seconds, less than 3 seconds, less than 2 seconds.

In another preferred embodiment, the amylopectin is not in the form of a starch or modified starch. Preferably, the amylopectin is purified. Preferably, the amylopectin does not contain amylose.

In another preferred embodiment, the solid dosage form is porous. More preferably, the solid dosage form is highly porous, at least 10%. More preferably, the solid dosage form has a porosity of greater than 60%. Preferably, the solid dosage form has voids in the micrometer size range that form a porous interconnecting network. Preferably, the solid dosage form comprises a porous interconnecting network and not a polymer that forms a dense continuous (non-porous) sheet.

In another preferred embodiment, the solid dosage form is not a film. Preferably, the solid dosage form does not comprise a water-soluble synthetic polymer as the primary matrix-forming agent.

In another preferred embodiment, the solid dosage form is not a tablet.

In another preferred embodiment, the solid dosage form is not a capsule.

In another preferred embodiment, the solid dosage form is not a lozenge.

In another preferred embodiment, the solid dosage form is not a standard release dosage form.

In another preferred embodiment, the solid dosage form is not a standard stomach-release dosage form.

In another preferred embodiment, the solid dosage form is not a normal fast release dosage form.

In another preferred embodiment, the solid dosage form is not a normal fast stomach-release dosage form.

In another preferred embodiment, the dosage form is not a liquid, or a solvent- or oil-based material.

In another preferred embodiment, the solid dosage form is lyophilised.

In another preferred embodiment, the matrix forming agents have at least one of the following properties: (i) dispersed throughout the structure, (ii) allows water molecules to diffuse out under vacuum to form a porous network, (iii) interacts with low molecular weight crystalline water soluble agents to form mostly amorphous three-dimensional structures, (iv) prevents crystallization of the active drug form (if it is initially dissolved) as it transfers to the solid state during lyophilisation, (v) has the ability to not be hygroscopic, (vi) has the ability to impart the physical strength to allow the dosage form to be expressed from packaging and handled with bare hands, and so on.

In another preferred embodiment, the dosage form is >96% water soluble and drug molecules are not trapped or bound to insoluble particles or colloids, but rather diffuse rapidly through a true solution. In another preferred embodiment, the matrix is >96% water soluble.

In another preferred embodiment, the dosage form matrix forming agents are >96% non-ionizable so that drug/matrix interactions will be minimised and drug/membrane interaction maximised. In another preferred embodiment, the matrix is >96% non-ionizable.

In another preferred embodiment, the disintegrated dosage form forms an imperceptible “bolus” under the tongue, which is viscous enough to stay in place for several minutes without draining away, but not too viscous so as to restrict the diffusion of the drug to the membrane unduly.

In another preferred embodiment, the solid dosage form requires a high surface area to volume to maximize rapid water contact with all parts of the dosage form due to capillary action, and maximises drug molecule diffusion into the sublingual membrane.

In another preferred embodiment, in the solid dosage form has at least one of the following properties: (1) hard, non-flexible, non-elastic, friable solid; (2) porous; (3) the API occupies void spaces; (4) upon contact with moisture, swells and then disintegrates and fragments from the inside outward, followed by dissolution of fragments; (5) formed by freeze drying; (6) almost completely dry (and <5% water); and (7) protected from absorbing water during storage.

In another preferred embodiment, the biologically active material is present in an amount from 0.02 to 95 weight % by dry weight of the composition of the dosage form.

Preferably, the dosage form is administered to the subject to deliver a dose of ketamine in the range of 0.1 mg to 150 mg/dosage form.

In another preferred embodiment, the depression is selected from the group consisting of: major depressive disorder or treatment-resistant depression. Preferably, the treatment-resistant depression is characterised by the inability of normal anti-depressants to be effective accompanied by a suicidal modality. Preferably, the treatment-resistant depression is characterised as major depressive disorder in a patient who do not respond to 2 separate trials of different antidepressants of adequate dose and duration in the current episode. Preferably, the depression is diagnosed by a physician as treatment-resistant depression. Preferably, the method substantially alleviates at least one symptom of the depression. Preferably, the treatment resistant depression failed at least two conventional antidepressant treatments. Preferably, the subject is diagnosed with treatment resistant depression failed at least two conventional antidepressant treatments. Preferably, the severity of the depression is scored using the Hamilton Depression Rating Scale. Preferably, the depression is diagnosed and assessed using the Brief Psychiatric Rating Scale.

In another preferred embodiment, the solid dosage form provides an effective plasma concentration of ketamine material within a period of no more than two hours, 30 minutes, 20 minutes, or 15 minutes. Preferably, the solid dosage form provides an effective plasma concentration of ketamine material within 15 minutes. Preferably, the solid dosage form comprises a dose of ketamine selected from the group consisting of: between 1 and 150 mg. Preferably, the solid dosage form comprises a dose of ketamine selected from the group consisting of: 25 mg, 50 mg, 75 mg, 100 mg, 125 mg and 150 mg. In another embodiment, the dosages are higher such as 175 mg, 200 mg, 225 mg, 250 mg, and 275 mg.

In another preferred embodiment, the solid dosage form provides a Cₘₐₓ at a comparable time to an IV injection but a lower concentration than that of an injection of the same dosage. Preferably the Cₘₐₓ is between 10 ng/ml⁻¹ and 200 ng/ml⁻¹, between 30 ng/ml⁻¹ and 150 ng/ml⁻¹ and between Between 50 ng/ml⁻¹ and 128.3 ng/ml⁻¹.

In another preferred embodiment, the solid dosage form provides a tₘₐₓ at a comparable time to an IV injection. Preferably, the solid dosage form provides a tₘₐₓ selected from the group consisting of: between 10 mins and 1 hour; between 25 minutes and 1 hour; between 15 minutes and 30 minutes; between 15 to 30 minutes; between 20 to 40 minutes; between 25 to 35 minutes; between 26 to 24 minutes; between 27 to 33 minutes; between 28 to 32 minutes; between 29 and 31 minutes; and 30 minutes.

In another preferred embodiment, the ketamine is rapidly absorbed with detectable concentrations at the first sampling time of 3 minutes. Preferably, the median time to peak plasma concentration of ketamine (tₘₐₓ) is reached at 30 minutes. Preferably, the ketamine absolute bioavailability is 29% with low variability. Preferably, the exposure to ketamine and norketamine enantiomers is approximately dose proportional using sublingual doses over the range 25-100 mg.

Preferably, the Area Under the Curve (AUC) is selected from the group consisting of: Between 50 and 500 ng/ml⁻¹ h; Between 150 and 250 ng/ml⁻¹ h and Between 161.6 and 211.3 ng/ml⁻¹ h.

Preferably the total dose of ketamine delivered to the patient is selected from the group consisting of: between 0.01 to 5 mg/kg; between 0.1 to 1 mg/kg and 0.5 mg/kg.

In another preferred embodiment, the dosage form is non-ionisable. Preferably, The dosage form matrix is >96% non-ionisable, more preferably >10%, more preferably >60%, more preferably 65-75%.

In another preferred embodiment, the dosage form is is greater than 96% water soluble.

In another preferred embodiment, the dosage form is administered to the subject utilising a dosing regimen selected from the group consisting of: at a frequency to alleviate the symptoms of depression, twice hourly, once every six hours, once every 12 hours, once daily, twice weekly, once weekly, once every two weeks, once a month, every two months, once every six months, once yearly. Preferably, the dosage form is administered to the subject twice weekly, and then decreases in frequency to once weekly or less. Preferably, the dosage form may be administered by the patient. Preferably, the dosage form may be administered without the immediate supervision of a physician or nurse. Preferably, the dosage form may be administered outside of a clinical setting. Preferably, the dosage form may be administered by the patient upon their decision.

In another preferred embodiment, the dosage form comprises matrix aide glycine. Preferably, glycine is present in an amount from 0.5 to 5 weight % by dry weight of the composition of the dosage form.

In another preferred embodiment, the dosage form comprises a lubricant. Preferably, the lubricant is polyethylene glycol (PEG) 800-30,000, preferably PEG 1500. Preferably, PEG 1500 is present in an amount from 0.05 to 5 weight % by dry weight of the composition of the dosage form.

In another preferred embodiment, dosage form further comprises a buffer reagent. Preferably, the buffer reagent comprises sodium carbonate. Preferably, sodium carbonate is present in an amount from 0.01 to 10 weight % by dry weight of the composition of the dosage form.

In another preferred embodiment, the dosage form comprises an absorption enhancer.

Preferably, the absorption enhancer comprises β-cyclodextrin. Preferably, β-cyclodextrin is present in an amount from 0.01 to 10 weight % by dry weight of the composition of the dosage form. Preferably, the dosage form comprises a flocculating agent. Preferably, the dosage form comprises a surfactant. Preferably, the dosage form comprises an additive. Preferably, the dosage form comprises a colouring agent. Preferably, the colouring agent is selected from the group consisting of colours compliant with pharmaceutical regulations, and mixtures therein. Preferably, the dosage form comprises a flavouring agent. Preferably, the flavouring agent is selected from flavours and sweeteners compliant with pharmaceutical regulations, and mixtures therein.

In another preferred embodiment, the dosage form comprises at least one pharmaceutically acceptable carrier.

In another preferred embodiment, the method does not comprise the administration of a further anti-depressant compound.

In another preferred embodiment, the method does comprise the administration of a further anti-depressant compound.

Preferably, the method comprises the administration of the ketamine wafer solid dosage form at the same time a further oral anti-depressant is commenced, so that ketamine's rapid anti-depressant effects can bridge the delay in onset of the oral therapy.

In another preferred embodiment, method address major depressive disorder with increased suicide risk, by incorporating adjunct ketamine therapy in any patient who displays increased suicidalty who is already on standard oral anti-depressant therapy.

In another preferred embodiment, the method further comprises the administration of a further anti-depressant compound. Preferably, the further anti-depressant compound is administered concurrently with, before or after the administration of the solid dosage form.

Preferably, the further anti-depressant compound forms part of the solid dosage form.

In a further aspect, the invention is a method for improving compliance with a ketamine prescription in a patient suffering depression, said method comprising the method described above. Preferably, the said method improves compliance by ensuring the patient takes the medication at the required time.

Note that disclosure of a range includes disclosure of each individual integer within that numerical range and includes up to 2 decimal points within the integer. Each individual integer is expressly disclosed within the range as presented herein.

TABLE 1
Further preferred aspects of the invention.

| | | Preferred | More Preferred | Most Preferred |
|---|---|---|---|---|
| | | Amounts (dry | Amount (dry | Amounts (dry |
| Type | Name | wafer weight) | wafer weight) | wafer weight) |
| Biologically | Ketamine | 0.1-45%, including | 5-45%, including | 5-45%, including |
| active material | | quantities | quantities | quantities |
| | | equating to a | equating to 5 mg, | equating to 5 mg, |
| | | ketamine dose of | 10 mg, 25 mg, | 10 mg, 25 mg, |
| | | dosages of 1 to | 50 mg, 75 mg, | 50 mg, 75 mg, |
| | | 100 mg, 1 to | 100 mg, 150 mg. | 100 mg, 150 mg. |
| | | 150 mg, including | | |
| | | 5 mg, 10 mg, | | |
| | | 25 mg, 50 mg, | | |
| | | 75 mg, 100 mg, | | |
| | | 150 mg. | | |
| Biologically | Ketamine | amorphous solid | XRD | |
| active material | | | spectrum | |
| | | | amorphous | |
| | | | ketamine | |
| | | | lacks peaks | |
| | | | due to | |
| | | | crystalline | |
| | | | ketamine. | |
| Matrix forming | Amylopectin | 2-17% | 8-14% | 8%, 9%, 10%, |
| agent | | | | 11%, 12%, 13%, |
| amorphous, | | | | 14%, 15%, 16%, |
| non-ionic | | | | or 17%, and within |
| | | | | 2 decimal points |
| | | | | of each figure. |
| Ionic matrix | Sodium | 0.1-4% | 1.5-2.5% | 1%, 2%, 3% or |
| aides | carboxymethyl | | | 4%, and within 2 |
| | cellulose | | | decimal points of |
| | (CMC) | | | each figure. |
| Ionic matrix | Glycine | 0.1-4% | 1.5-2.5% | 1%, 2%, 3% or |
| aides | | | | 4%, and within 2 |
| | | | | decimal points of |
| | | | | each figure. |
| Non-ionic | PEG 1500 | 0.1-10% | 1.5-3.5% | 1%, 2%, 3%, 4% |
| matrix aides | | | | or 5%, and within |
| | | | | 2 decimal points |
| | | | | of each figure. |
| Non-ionic | microcrystalline | 0.1-10% | 1.5-3.5% | 1%, 2%, 3%, 4%, |
| matrix aides | cellulose | | | 5%, 6%, 7% 8%, |
| | (MCC) | | | 9% or 10% and |
| | | | | within 2 decimal |
| | | | | points of each |
| | | | | figure. |
| Matrix forming | Mannitol | 0-80% | 30-50% | 35%, 36%, 37%, |
| agent | | | | 38%, 39%, 40%, |
| crystalline, | | | | 41%, 42%, 43%, |
| non-ionic | | | | 44%, or 45% and |
| | | | | within 2 decimal |
| | | | | points of each figure. |
| Matrix forming | Lactose | 0-80% | 10-30% | 25%, 26%, 27%, |
| agent | | | | 28%, 29%, 30%, |
| crystalline, | | | | 31%, 32%, 33%, |
| non-ionic | | | | 34%, or 35% and |
| | | | | within 2 decimal |
| | | | | points of each |
| | | | | figure. |
| pH | pH | pH 3.0 to 8.0 | pH 5 to 6 | pH 5 to 6 |
| Buffer reagent | Sodium | 0-10% | 0.1-0.3% | 0.1, 0.2, 0.3, 0.4, |
| | carbonate | | | 0.5%% |
| Coloring agent | Pharmaceutically | 0-1% | qs | qs |
| | acceptable | | | |
| | colouring | | | |
| | agent | | | |
| Flavouring | Pharmaceutically | 0-5% | qs | qs |
| agent | acceptable | | | |
| | flavouring | | | |
| | agent and/or | | | |
| | sweetener | | | |
| Crystallinity | | powder x-ray | powder x-ray | powder x-ray |
| factor of | | diffraction (XRD) | diffraction (XRD) | diffraction (XRD) |
| dosage form | | of the dosage | of the dosage | of the dosage |
| | | form comprises | form comprises | form comprises |
| | | peaks at 2-theta | peaks at 2-theta | peaks at 2-theta |
| | | values at | values at | values at |
| | | approximately | approximately | approximately |
| | | 9.58 degrees, | 9.58 degrees, | 9.58 degrees, |
| | | 19.68 degrees, | 19.68 degrees, | 19.68 degrees, |
| | | and 20.05 | and 20.05 | and 20.05 |
| | | degrees; those for | degrees; those for | degrees; those for |
| | | crystalline | crystalline | crystalline |
| | | ketamine or its | ketamine or its | ketamine or its |
| | | salts are | salts are | salts are |
| | | substantively | substantively | substantively |
| | | absent | absent | absent |
| Disintegration | Dosage form | <60 s; or <30 s | <60 s | <60 s, <20 s, <10 |
| rate | disintegrates | | | s, <5 s |
| | once placed | | | |
| | in the oral | | | |
| | cavity in a | | | |
| | time period. | | | |
| Dissolution | Dosage form | <60 seconds after | <60 seconds after | <60 seconds after |
| rate | dissolves | disintegration | disintegration | disintegration |
| | once placed | or <30 seconds after | | |
| | in the oral | disintegration | | |
| | cavity in a | | | |
| | time period. | | | |
| Solubility | The dosage | | | |
| | form is | | | |
| | greater than | | | |
| | 96% water | | | |
| | soluble. | | | |
| Non-ionisable | The dosage | >10% | >60% | >90%, 65-75%. |
| | form matrix | | | |
| | is >96% | | | |
| | non- | | | |
| | ionisable | | | |
| Porosity | Proportion of | >10% | >60% | >60%, 65-75%. |
| | water | | | |
| | removed by | | | |
| | lyophilization | | | |
| Dose (mg of | | 0.01 to 5 mg/kg | 0.1 to 1 mg/kg, | 0.5 mg/kg |
| ketamine/kg) | | | | |
| Tₘₐₓ | The | Between 10 mins | Between 25 | Between 15 |
| | detectable | and 1 hour; | minutes and 1 | minutes and 30 |
| | levels of | | hour. | minutes |
| | ketamine in | | | |
| | the blood is | | | |
| | measured | | | |
| | after several | | | |
| | minutes, and | | | |
| | tₘₐₓ is | | | |
| | observed | | | |
| | between a | | | |
| | range of | | | |
| | minutes, | | | |
| | comparable | | | |
| | to IV. | | | |
| Cₘₐₓ | ng/ml⁻¹ | Between 10 | Between 30 | Between 50 |
| | | ng/ml⁻¹ and | ng/ml⁻¹ and | ng/ml⁻¹ and |
| | | 200 ng/ml⁻¹ | 150 ng/ml⁻¹ | 128.3 ng/ml⁻¹ |
| Area Under the | ng/ml⁻¹ h | Between 50 and | Between 150 and | Between 161.6 and |
| Curve (AUC) | | 500 ng/ml⁻¹ h | 250 ng/ml⁻¹ h | 211.3 ng/ml⁻¹ h |

### BRIEF DESCRIPTION OF THE DRAWINGS

FIG. 1 represents the disintegration apparatus (all dimensions are expressed in mm).

FIG. 2 demonstrates the absolute bioavailability study of Wafermine 25 mg (KET012 Study).

FIG. 3 demonstrates the predicted NMDA receptor occupancy post IV ketamine infusion (Zarate 2006).

FIG. 4 demonstrates the predicted NMDA receptor occupancy post Wafermine 100 mg SL administration.

FIG. 5 provides a summary of ketamine and norketamine concentrations after single dose (50-100 mg) administration, based on the study KET012 population PK model.

FIG. 6 provides a summary of ketamine and norketamine concentrations after single dose (125-200 mg) administration, based on the study KET012 population PK model.

FIG. 7 provides a summary of ketamine and norketamine concentrations after single dose (225-275 mg) administration, based on the study KET012 population PK model.

FIG. 8 provides simulated ketamine concentrations over time, stratified on dose, after single dose administration. The figure shows the median concentration, as well as the 5th, the 25th, the 75th and the 95th percentiles of simulated data.

FIG. 9 provides simulated norketamine concentrations over time, stratified on dose, after single dose administration. The figure shows the median concentration, as well as the 5th, the 25th, the 75th and the 95th percentiles of simulated data.

FIG. 10 provides individual racemic ketamine plasma concentration—time curves and geo-metric mean (bold line) during the first 12 h following a 10 mg dose given during a 30 min i.v. infusion to eight healthy volunteers.

FIG. 11 provides individual racemic ketamine plasma concentration—time profiles and geometric mean (bold line) during the first 12 h following a 25 mg sublingual dose to eight healthy volunteers.

FIG. 12 provides geometric mean racemic ketamine plasma concentration— time profiles during the first 6 h following sublingual administration of 25 mg (continuous line) and 10 mg as a 30 min i.v. infusion (dashed line) to eight healthy volunteers.

### DETAILED DESCRIPTION OF THE INVENTION

### General

Those skilled in the art will appreciate that the invention described herein is susceptible to variations and modifications other than those specifically described. It is to be understood that the invention includes all such variations and modifications. The invention also includes all of the steps, features, compositions and materials referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The invention described herein may include one or more ranges of values (e.g. size, concentration etc). A range of values will be understood to include all values within the range, including the values defining the range, and values adjacent to the range that lead to the same or substantially the same outcome as the values immediately adjacent to that value which defines the boundary to the range.

The entire disclosures of all publications (including patents, patent applications, journal articles, laboratory manuals, books, or other documents) cited herein are hereby incorporated by reference. Inclusion does not constitute an admission is made that any of the references constitute prior art or are part of the common general knowledge of those working in the field to which this invention relates.

Throughout this specification, unless the context requires otherwise, the word “comprise” or variations, such as “comprises” or “comprising” will be understood to imply the inclusion of a stated integer, or group of integers, but not the exclusion of any other integers or group of integers. It is also noted that in this disclosure, and particularly in the claims and/or paragraphs, terms such as “comprises”, “comprised”, “comprising” and the like can have the meaning attributed to it in US Patent law; e.g., they can mean “includes”, “included”, “including”, and the like.

“Therapeutically effective amount” as used herein with respect to methods of treatment and in particular drug dosage, shall mean that dosage that provides the specific pharmacological response for which the drug is administered in a significant number of subjects in need of such treatment. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that drug dosages are, in particular instances, measured as oral dosages, or with reference to drug levels as measured in blood. Amounts effective for such a use will depend on: the desired therapeutic effect; the potency of the ketamine material; the desired duration of treatment; the stage and severity of the disease being treated; the weight and general state of health of the patient; and the judgment of the prescribing physician.

Major depressive disorder (MDD) is a mental disorder characterized by at least two weeks of low mood that is present across most situations. It is often accompanied by low self-esteem, loss of interest in normally enjoyable activities, low energy, and pain without a clear cause.

Treatment Resistant Depression (TRD) is characterised by the inability of normal anti-depressants to be effective accompanied by a suicidal modality. According to the FDA, TRD is a MDD in a patient who does not respond to 2 separate trials of different antidepressants of adequate dose and duration in the current episode.

Ketamine in low doses has been found to be a potentially effective therapy. However, treatment requires travel to a clinic and administration by intravenous (IV) injection. Infusion takes less than one hour; however the patient needs to be monitored at the clinic for some time afterwards. This burdensome and lengthy protocol may discourage depressed patients from seeking help in time, or at all.

In one preferred embodiment, the invention provides a solution to counteract these issues, and allows an almost immediate patient-decided (with distant real-time on-line clinical advice) administration of ketamine in physiologically relevant concentrations without travel, IV or a clinical setting.

Ketamine is generally unsuitable in oral form when required for fast-acting purposes such as the treatment of suicidal depression or acute pain. In one preferred embodiment, the invention provides a solution to that problem.

One of the problems to be solved was the requirement to deliver ketamine in physiologically useful concentrations (Cₘₐₓ) directly to the bloodstream (like IV), rapidly in an oral form, yet replicate the fast-acting (short tₘₐₓ) effects of IV required by the time-critical nature of the depressive condition. In one preferred embodiment, the invention provides a solution to the problem by combining a series of properties into a solid dosage form so as to achieve all these outcomes for the first time, delivered over a period of minutes, without the need for the patient making a decision to travel, making the journey, seeing a clinician in a clinical setting, and receiving treatment, a period of hours.

In one preferred embodiment, the invention comprises a solid sublingual (SL) oral dosage form where all of these steps combine to achieve the objective:
the ketamine is present at between 1 and 200 mg;
the ketamine is in the form of an amorphous solid distributed throughout the dosage form so that it dissolves instantly on contact with saliva;
the dosage form is highly porous (greater than 60% porosity) so that contacting saliva is drawn into the centre of the dosage form for immediate dissolution;
despite the porosity, the dosage form is robust to allow the patient to dispense and hold the dosage without breaking;
the dosage form in place disintegrates within 60 s to allow rapid ketamine SL absorption;
the dosage form matrix is greater than 96% water soluble so prevent ketamine being trapped in solid or colloidal particles;
the dosage form matrix is >96% non-ionisable, to prevent ketamine being complexed to the matrix and not travel to the sublingual membrane;
the property of the matrix being non-ionizable allows the pH of the matrix to be adjusted to optimise the diffusion of ketamine through the SL membrane;
the detectable levels of ketamine in the blood, as a result of these combinations, is measured after several minutes, and tₘₐₓ is observed between 15-30 minutes, comparable to IV;
further, Cₘₐₓ and the Area Under the Curve (AUC) are physiologically relevant and useful.

The precise quantity of ketamine present in the composition is generally present in an amount from 0.02 to 95%, preferably 0.02 to 20% or preferably 0.1 to 75%, 1 to 45% by dry weight of the composition of the dosage form.

The fast dissolving solid dosage form of the present invention also comprises at least one matrix forming agent. In the freeze-dried systems of the prior art, gelatin is the most commonly used carrier or structure forming agent due to its wall-forming ability. Gelatin is an ionic water soluble polymer, and as such, when mixed with active pharmaceutical ingredients in water; the increasing viscosity of the solution over time may cause a decreasing solubility of poorly soluble drugs in the mixture, and lead to a suspension of the drug in gelatin matrix. This can cause phase separation to occur; and the drug in amorphous or crystalline forms may not be homogenously dispersed in the matrix, which will eventually affect the dissolution and absorption of the final product.

Applicant has found that other polymer materials suitable for forming a matrix may be selected for specific application in the field of drug delivery, especially for site-specific drug delivery system such as in the oral cavity. Matrix forming agents of the present invention may be selected from the group consisting of: non-mammalian gelatin, dextrin, soy protein, wheat protein, psyllium seed protein, acacia gum, guar gum, agar gum, xanthin gum, polysaccharides; alginates; sodium carboxymethylcellulose; carrageenans; dextrans; pectins; sugars; amino acids; starch; modified starches; carboxymethylcellylose; hydroxypropylmethylcellulose; hydroxypropyl cellulose and methyl cellulose inorganic salts; synthetic polymers; amylopectin, polypeptide/protein or poly-saccharide complexes. Examples of at least one matrix forming agent that are carbohydrates include mannitol, dextrose, lactose, galactose, sorbitol and trehalose and cyclodexrin. Examples of matrix forming agents that are inorganic salts may be selected from the group consisting of: sodium phosphate, sodium chloride and aluminium silicates. The at least one matrix forming agent may also be an amino acid. Examples of suitable amino acids include glycine, L-alanine, L-aspartic acid, L-glutamic acid, L-hydroxyproline, L-isoleucine, L-leucine and L-phenylalanine.

In a highly preferred embodiment, at least one matrix forming agent is sodium carboxymethylcellulose. When at least one matrix forming agent is sodium carboxymethyl cellulose, the polymer is present in a concentration of from about 0.1% to about 19% by dry weight of the solid dosage form. In a preferred embodiment the sodium carboxymethylcellulose is present in an amount of about 0.1% to about 15% by dry weight of the dosage form. In a highly preferred embodiment of the present invention, the sodium carboxymethyl cellulose is present in an amount of about 0.1% to about 1.0% by dry weight of the solid dosage form. In another embodiment of the present invention, the fast dissolving dosage form comprises amylopectin as at least one matrix forming agent. Amylopectin is capable of increasing the release of ketamine by promoting formulation disintegration. Amylopectin may be present in the dosage form at a concentration about 2% up to no great than 20% by dry weight of the solid dosage form. In a highly preferred form of the present invention, amylopectin is present in an amount of about 2% to about 17% dry weight of the dosage form.

To achieve a rapid dissolution of drugs, low MW diluents may be added as at least one matrix forming material. Diluents include microcrystalline cellulose (e.g., Avicel PH 101® and Avicel PH 102®), lactose, starch and sorbitol. These diluents may be present in the dosage form either alone or as a mixture in different ratios, and may be about 1% to about 80%, preferably about 2% to about 50%, either individually or cumulatively. In one embodiment of the present invention, the fast dissolving dosage form comprises microcrystalline cellulose as the at least one matrix forming agent. Microcrystalline cellulose may act as a filler and binder in the dosage form of the present invention. Microcrystalline cellulose has the ability to compact with minimum compression pressures, and results in a hard, stable and fast dissolving dosage form. Due to its large surface area and high internal porosity, microcrystalline cellulose is able to absorb and retain large amounts of water, which is desirable in the dosage form of the invention. When the solid dosage form of the present invention comprises microcrystalline cellulose, it is present in an amount of about 1% to about 10%, and preferably from about 1% to about 8% by dry weight of the dosage form. The effectiveness of the fast dissolving dosage form of the present invention relies on the drug dissolving in a small volume of fluid, such as in the oral cavity, prior to absorption into the systemic circulation. Therefore, the rate of dissolution of the dosage form is important. In a preferred embodiment of the present invention, the dosage form comprises a super-disintegrant as at least one matrix forming material.

In a highly preferred embodiment, the fast dissolving dosage form of the present invention comprises glycine. Glycine is an amino acid with excellent wetting properties and is suitable for the fast dissolving formulation. Low amounts of glycine may be used in the formulation of the present invention to control the dissolution rate of the dosage form. Furthermore, glycine may also be used as an anti-collapsing agent, which maintains the dosage form from shrinking either during the manufacture process or after packing. In one embodiment, the dosage form of the present invention comprises from about 0.5% to about 5% dry weight of the dosage form. According to another embodiment of the invention, the fast dissolving solid dosage form may include a matrix forming agent such as mannitol. Mannitol is a component that may aid in the crystalline structure and impart hardness of the dosage form. When mannitol is present in the dosage form, it occurs in a concentration of from about 5% to about 80%, and preferably from about 10% to about 60% by dry weight of the dosage form.

In addition, the fast dissolving dosage form of the present invention may include lubricants, such as polyethylene glycol (PEG) 1000, 1500, 2000, 4000 and 6000, sodium lauryl sulphate, fats or oils. One advantage of the use of these lubricants is to aid in the removal of the dosage form from the mould. These lubricants may be present in the dosage form either alone or as a mixture in different ratios, and may be between 0.05% to 5%, preferable between 0.1% and 2%, preferable about 1.5%, either individually or cumulatively. In one embodiment, the composition includes between 0.05% to 5% polyethylene glycol 1500, preferably between 0.1% and 2% by dry weight of the dosage form, or as mixtures of the various glycols. The invention extends, in another aspect thereof, to improve sublingual absorption of weak base compounds, the composition comprising a solid buffer reagent that affords to produce a saliva pH of 4-6 when dissolved in oral cavity. Increasing the pH of the solution of a weak base compound can increase the ratio of unionized to ionized,

Which will lead to enhanced sublingual absorption. The solid buffer reagent include sodium dihydrogen phosphate dihydrate, sodium hydrogen phosphate, sodium hydrogen carbonate and sodium carbonate, which may be present in the dosage form either alone or as a mixture in different ratios in a concentration of about 0.01% to about 10% by weight of the composition. Preferably, the buffer reagent is sodium carbonate, which may be present in a concentration of about 0.01% to about 10% by weight of the composition, preferably between 0.1% to 1%, most preferably about 0.3%.

When mannitol is present in the dosage form, it occurs in a concentration of from about 5% to about 80%, and preferably from about 10% to about 60% by dry weight of the dosage form.

The composition may, in certain embodiments, include an absorption enhancer. The absorption enhancer may be a polysaccharide and may be positively charged. Preferably, the absorption enhancer is β-cyclodextrin or its derivatives. The β-cyclodextrin or derivative may be present in a concentration of from about 0.01% to about 10% by dry weight of the dosage form, preferably between 0.2% to 2%, and most preferably about 1%. The fast dissolving solid dosage form of the present invention may comprise flocculating agents to maintain disbursement of ketamine evenly dispersed in the matrix during the manufacture process. The flocculating agent may be gums. Preferable, the gum is xanthan gum. The xanthan gum may be present in a concentration of about 0.01% to about 10% by dry weight of the composition, preferably from about 0.2% to 2%, and most preferably about 1%.

To aid dissolution of the ketamine into the aqueous environment, a surfactant may be added to the solution as a wetting agent. Suitable surfactants include anionic detergents such as sodium lauryl sulfate, dioctyl sodium sulfosuccinate and dioctyl sodium sulfonate. Cationic detergents may be used and include benzalkonium chloride or benzethomium chloride. The list of possible non-ionic detergents includes lauromacrogol 400, polyoxyl 40 stearate, polyoxyethylene hydrogenated castor oil 10, 50 and 60, glycerol monostearate, Polysorbate 40, 60, 65 and 80, sucrose fatty acid ester, methyl cellulose and carboxymethyl cellulose. These surfactants may be present in the dosage form either alone or as a mixture in different ratios. Additives which potentially enhance uptake of the compounds are fatty acids such as oleic acid, linoleic acid and linolenic acid.

In order to enhance the aesthetic and taste appeal of the fast dissolving dosage form to the subject, the dosage form may also contain colouring agents, such as FD & C dyes Blue No. 2 and Red No. 40; flavoring agents, such as orange, mixed berry, cherry, peppermint, raspberry and caramel; and/or sweeteners such as aspartame, stevia, sucralose and saccharin.

The fast dissolving solid dosage form of the present invention is suitable for oral administration to a subject. As discussed above, the dosage form comprises ketamine. The ketamine is therefore delivered to the subject via the oral cavity mucosa and into the systemic blood system within a relatively short period of time. In a preferred embodiment, an effective plasma concentration of the ketamine is reached within a period of no more than two hours, preferable within 30 minutes, and most preferably within 10 minutes.

Furthermore, an advantage of the present invention is that the fast dissolving solid dosage form completely dissolves within 2 seconds to 60 seconds, preferably 2 seconds to 30 seconds, and most preferably within 2 seconds to 10 seconds after administration of the dosage form. In a highly preferred embodiment of the present invention, there is no residue remaining of the dosage form of the present invention after administration that is detectable by the patient. As such, the subject has no urge to swallow the dosage form.

The subject receiving the fast dissolving dosage form of the present invention may be an animal or human being. When the subject is a human being, it may be an adult or a child, including elderly adults and infants. In particular the subject is a subject that is unable to or has difficulties in swallowing.

The fast dissolving solid dosage form may comprise sodium carboxymethylcellulose as a formulation aide in low levels. When the amount of sodium carboxymethylcellulose is between about 0.1% and 15% by dry weight of the dosage form, the wafer releases the active agent rapidly, without leaving a residue in the oral cavity. In addition, the use of gelatin was avoided by the inventors, and therefore prevents the unwanted residue left in the oral cavity after administration. The addition of lactose and or mannitol was also found to be advantageous in the dosage formulation of the present invention.

Thus, in one embodiment, the present invention provides a rapidly dissolving solid dosage form adapted for the release of ketamine in the oral cavity wherein the dosage form comprises: (i) ketamine and (ii) at least one matrix forming agent, wherein the dosage form substantially dissolves in the oral cavity, wherein the dosage form comprises 0.1-0.3% sodium carbonate, 0.1-4% sodium carboxymethylcellulose, 0.1-10% PEG 1500, 1-4%% glycine, 1-10%% microcrystalline cellulose; 2-17% amylopectin, 10-30% lactose and 30-50% mannitol as a dry weight of the solid dosage form, and which does not result in substantial detectable levels of residue left over in the oral cavity of the patient.

As discussed above, the medicaments of the present invention may include one or more pharmaceutically acceptable carriers. The use of such media and agents for the manufacture of medicaments is well known in the art. Except insofar as any conventional media or agent is incompatible with the pharmaceutically acceptable material, use thereof in the manufacture of a pharmaceutical composition according to the invention is contemplated. Pharmaceutical acceptable carriers according to the invention may include one or more of the following examples:

(1) surfactants and polymers, including, however not limited to polyethylene glycol (PEG), polyvinylpyrrolidone, polyvinylalcohol, crospovidone, polyvinylpyrrolidone-polyvinylacrylate copolymer, cellulose derivatives, hydroxypropylmethyl cellulose, hydroxypropyl cellulose, carboxymethylethyl cellulose, hydroxypropylmethyl cellulose phthalate, polyacrylates and polymethacrylates, urea, sugars, polyols, and their polymers, emulsifiers, sugar gum, starch, organic acids and their salts, vinyl pyrrolidone and vinyl acetate; and/or
(2) binding agents such as various celluloses and cross-linked polyvinylpyrrolidone, microcrystalline cellulose; and/or (3) filling agents such as lactose monohydrate, lactose anhydrous, microcrystalline cellulose and various starches; and/or
(4) lubricating agents such as agents that act on the increased ability of the dosage form to be ejected from the packaging cavity, and/or
(5) sweeteners such as any natural or artificial sweetener including sucrose, xylitol, sodium saccharin, cyclamate, aspartame, and acesulfame K; and/or
(6) flavouring agents; and/or
(7) preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quaternary compounds such as benzalkonium chloride; and/or
(8) buffers; and/or
(9) diluents such as pharmaceutically acceptable inert fillers, such as microcrystalline cellulose, lactose, dibasic calcium phosphate, saccharides, and/or mixtures of any of the foregoing; and/or
(10) wetting agents such as corn starch, potato starch, maize starch, and modified starches, croscarmellose sodium, crosspovidone, sodium starch glycolate, and mixtures thereof; and/or
(11) disintegrants; and/or
(12) effervescent agents such as effervescent couples such as an organic acid (e.g., citric, tartaric, malic, fumaric, adipic, succinic, and alginic acids and anhydrides and acid salts), or a carbonate (e.g. sodium carbonate, potassium carbonate, magnesium carbonate, sodium glycine carbonate, L-lysine carbonate, and arginine carbonate) or bicarbonate (e.g. sodium bicarbonate or potassium bicarbonate); and/or
(13) other pharmaceutically acceptable excipients.

Medicaments of the invention suitable for use in animals and in particular in human beings typically must be sterile and stable under the conditions of manufacture and storage. The medicaments of the invention comprising ketamine can be formulated as a solid, a liposome, or other ordered structures suitable to high drug concentration adapted for oral delivery.

In another embodiment, the ketamine may be combined into a medicament with another biologically active material, or even the same biologically active material.

Medicaments of the invention can be orally administered to a subject. Solid dosage forms for oral administration include wafers, capsules, tablets, pills, powders, pellets, films and granules. Further, incorporating any of the normally employed excipients, such as those previously listed, and generally 0.1% to 95% of the ketamine, and more preferably at a concentration of 0.1% to 75% will form a pharmaceutically acceptable non-toxic oral administration.

According to a further aspect of the present invention, there is provided a method to produce the fast dissolving dosage form of the present invention comprising the steps of combining at least one matrix forming agent with a ketamine to form a mixture and then freeze drying the mixture to form the solid dosage form. In a preferred embodiment of the present invention, the mixture is measured (by weight or volume) into a preformed plastic or aluminium blister mould (individual dose). The blister mould is placed into a freeze dryer for 24 hours and the resultant solid dosage form (wafer) is then sealed with aluminium or plastics foil to prevent moisture absorption.

In one embodiment of the present invention, the method may require that the pH of the mixture is adjusted to a pH within the range of between 3.0 and 8.0, preferably between 6.4 and 7.8. If required, the pH may be adjusted by using an acid, such as hydrochloric acid, phosphoric acid or citric acid; or a basic compound such as sodium hydroxide, sodium dihydrogen phosphate dehydrate, sodium hydrogen phosphate, sodium hydrogen carbonate and sodium carbonate.

In another embodiment, the method may include the step of using a solvent, such as water. If water is used as a solvent, it is preferable to be removed by freeze drying.

In a further aspect of the present invention, there is provided a kit comprising the fast dissolving oral dosage form wherein the dosage form comprises: (i) ketamine, and (ii) at least one matrix forming agent, wherein the dosage form substantially dissolves in the oral cavity, and instructions for its use.

The present invention will now be described with reference to the following non-limiting Examples. The description of the Examples is in no way limiting on the preceding paragraphs of this specification, however is provided for exemplification of the methods and compositions of the invention.

### Wafermine for Depression

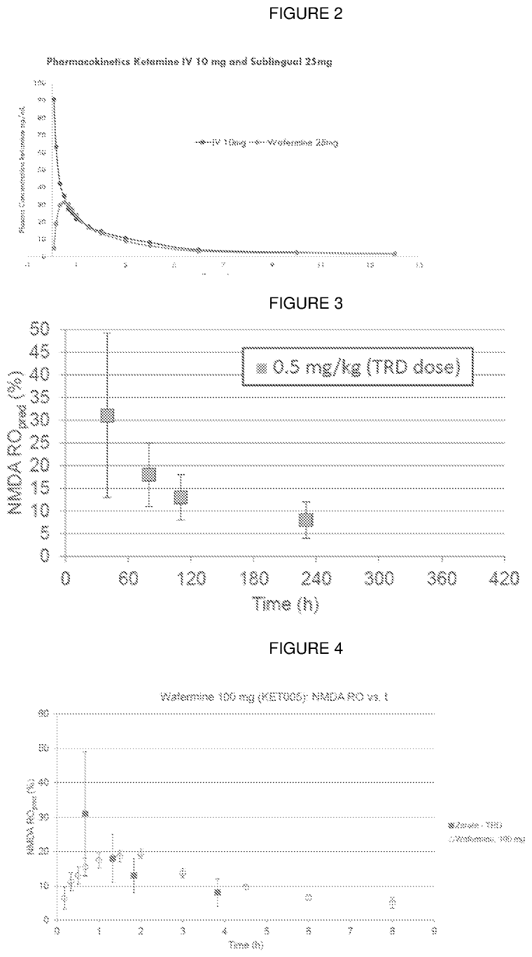

Wafermine has a number of potential advantages over alternative approaches:
(a) Sublingual administration may be associated with a better adverse event profile over IV and IN administration given the more gentle absorption profile avoids excessively high plasma ketamine concentrations post administration. Refer to FIG. 2 demonstrating the absolute bioavailability study of Wafermine 25 mg (KET012 Study). The study demonstrates the absence of the excessively high initial plasma ketamine concentrations with SL Wafermine compared to IV administration.
(b) Wafermine contains racemic ketamine, the enantiomeric mixture of (R)-ketamine and (S)-ketamine which, in one preferred embodiment, is more effective than (S)-ketamine alone. The superiority of (R)-ketamine does not seem to be related to a U-shaped dose response of the drugs, as it has been shown superior to (S)-ketamine with up to a 30-fold range of doses in multiple mouse tests of antidepressant efficacy.
(c) Sublingual administration is non-invasive (unlike IV) and typically better tolerated than IN administration. Wafermine is easy to use and convenient to store.
(d) Sublingual administration is usually associated with less variability in drug absorption over IN administration potentially resulting in more predictable clinical effect.

Refer to FIG. 3 and FIG. 4 which display predicted NMDA receptor occupancy following IV ketamine infusion (0.5 mg/kg over 40 mins) and Wafermine 100 mg SL, respectively. FIG. 3 demonstrates the predicted NMDA receptor occupancy post IV ketamine infusion (Zarate 2006). FIG. 4 demonstrates predicted NMDA receptor occupancy post Wafermine 100 mg SL administration.

The above modelling supports Wafermine SL can achieve adequate NMDA receptor occupancy which is postulated in a preferred embodiment as one of the main mechanisms by which ketamine exerts its anti-depressant effect.

A dosing range for Wafermine between 25 to 200 mg SL administered in single or divided doses, is likely to be able to achieve a clinically meaningful anti-depressant effect. In one embodiment, this dose may be administered once or twice weekly. In one preferred embodiment, dosing typically starts twice weekly, and then decreases in frequency to once weekly or less.

In summary, the invention provides a solution to current clinical disadvantages in the use of ketamine to treat depression and in particular, treatment resistant depression and major depressive disorder. Ketamine in low doses has been found to be a potentially effective therapy against depression. However, treatment requires travel to a clinic, during clinic hours, and administration over several hours by intravenous (IV) injection. This involved and lengthy protocol may discourage depressed patients from seeking help in time, or at all. This invention describes a solution to counteract these issues, and allow a portable, rapid-acting patient-chosen (with real-time on-line clinical advice) administration of ketamine in physiologically relevant concentrations without IV or a clinical setting. Ketamine is generally unsuitable in oral form when required for fast-acting purposes such as suicidal depression, and elsewhere acute pain, and analgesia. This invention solves that problem. The invention delivers ketamine in physiologically useful concentrations (Cₘₐₓ) directly to the bloodstream (like IV), rapidly in an oral form, yet replicates the fast-acting (short tₘₐₓ) effects of IV required by the time-critical nature of the depressive condition. The invention achieves this without the need for the patient making a decision to travel, making the journey, seeing a clinician in a clinical setting, and receiving treatment for a period of hours.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The present invention will now be described with reference to the following non-limiting Examples. The description of the Examples is in no way limiting on the preceding paragraphs of this specification, but is provided for exemplification of the methods and compositions of the invention.

### EXAMPLES

It will be apparent to persons skilled in the milling and pharmaceutical arts that numerous enhancements and modifications can be made to the above described processes without departing from the basic inventive concepts. For example, in some applications the biologically active material may be pretreated and supplied to the process in the pretreated form. All such modifications and enhancements are considered to be within the scope of the present invention, the nature of which is to be determined from the foregoing description and the appended claims. Furthermore, the following Examples are provided for illustrative purposes only, and are not intended to limit the scope of the processes or compositions of the invention.

### Example 1—Disintegration Profile of Wafermine

### Determination of Disintegration Times for Ketamine Wafers

Disintegration times of ketamine dosage forms of this invention, in the form of wafers, were determined according to standard methods in the art and according to the USP Monograph <701> Disintegration, sub-category Procedure and Criteria for Buccal Tablets, Sublingual Tablets, Capsules, Tablets for Oral Suspension, Tablets for Oral Solution, Tablets for Topical Solution, Orally Disintegrating Tablets, and Chewable Tablets.

### Experimental Setup

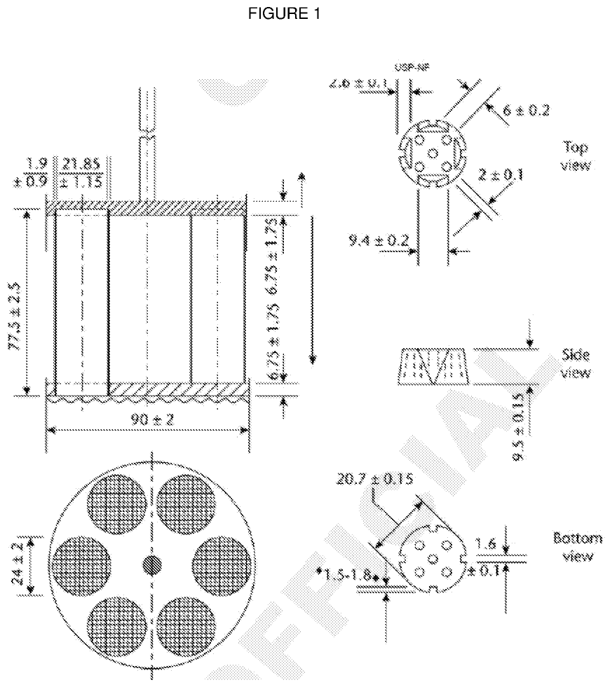

A diagram of the apparatus from the Monograph is replicated in FIG. 1. The apparatus consists of a basket-rack assembly, a 1000-mL low-form beaker 138 to 160 mm in height and having an inside diameter of 97 to 115 mm for the immersion fluid (pure water), a thermostatic arrangement for heating the water between 35° and 39°, and a device for raising and lowering the basket in the water at a constant frequency rate between 29 and 32 cycles per minute through a distance of NLT 53 mm and NMT 57 mm. The volume of the water in the vessel is such that at the highest point of the upward stroke the wire mesh remains at least 15 mm below the surface of the water and descends to NLT 25 mm from the bottom of the vessel on the downward stroke.

### Methodology

Pure water was used as the immersion medium, equilibrated at a temperature of 37° C.

Ketamine dosage forms of this invention, in the form of wafers, utilizing components described in Table 1, were placed into each of the 6 tubes of the basket-rack assembly. The apparatus was operated, using water as the immersion fluid, and maintained at 37±2°. The observed time for disintegration for each wafer was recorded. An average of all six wafers was reported. At the end of the time limit specification for disintegration, which is 30 seconds, the basket-rack assembly was lifted from the fluid, and the wafers observed by the technician.

### Results

When the above-described method is followed, all the wafers disintegrated completely and under 30 seconds. This experiment demonstrates that ketamine dosage forms of this invention disintegrate once placed in the oral cavity of a subject and in under 60 seconds.

### Example 2—a Single Dose of Wafermine can Treat Treatment-Resistant Major Depressed Patients—a Planned Study

The following study is planned. A double blinded trial of the Wafermine treatment for treatment-resistant major depressed patients will be conducted in 60 trial subjects, all of whom give informed consent to the trial. Subjects are randomised to Wafermine or placebo and also commence treatment with an oral anti-depressant not previously trialed by the patient. Spravato is also used as a positive control.

All the subjects recruited will display treatment resistant depression (who failed at least two conventional antidepressant treatments) and as diagnosed by standard methods in the art and the severity of the depression is scored using the Hamilton Depression Rating Scale (HAM-D), which is widely known and used in the field. The trial uses the Montgomery-Asberg Depression Rating Scale (MADRS) as primary endpoint. The primary endpoint is changed from baseline in MADRS at 4 weeks.

A clinical assessment of the subjects' symptoms is made upon entry into the study and every hour for 24 hours after treatment and then daily for 7 days. Less frequent assessment is preferable to improve data quality from avoiding patient fatigue and anchoring of responses. The clinical assessment includes the assessment of the treatment-resistant depression and symptoms according to the Brief Psychiatric Rating Scale (BPRS), widely known and used in the field.

Wafermine is prepared according to aspects of the invention described herein. Four Wafermine dosage forms is decided and chosen from the following total ketamine dosages: 25 mg, 50 mg, 75 mg, 100 mg, 150 mg and 0 mg (placebo), plus any positive control if required. The subjects are separated into 4 groups: Group 1—25-50 mg, Group 2—50-100 mg, Group 3—100-150 mg, Group 4—placebo, Group 5—positive control. Each group receives a single or multiple sublingual dose of Wafermine immediately upon the onset of depression.

Based on the information described here, all three single doses of Wafermine, at 25 mg, 50 mg and 75 mg ketamine, administered singly or multiply, will treat the depression and reduce the symptoms of the depression when compared to the placebo group. The results will be statistically significant.

This is supported by a number of IV studies. Firstly, Zarate et al demonstrated in a randomised clinical trial that a single IV dose of ketamine at 0.5 mg/kg caused anti-depressive effects in patients as measured by MADRS. Secondly, Wang et al demonstrated the that non-melancholic or anxious depression patients are effectively treated by 6 repeated IV doses of ketamine at 0.5 mg/kg as measured by MADRS. Thirdly, Bahji et al conducted metadata analysis and demonstrated that a single dose of IV ketamine at 0.5 mg/kg is more efficacious than intransal esketamine for the treatment of depression. However, it was not obvious that a single sublingual dose of a ketamine dosage form, as formulated and defined here, would achieve the requisite pharmacokinetic profile and effectively treat the depression.

### REFERENCES

1. Zanos P and Gould T D. Mechanisms of ketamine action as an antidepressant. Molecular Psychiatry (2018) 23, 801-811.
2. Dwivedi Y. Brain-derived neurotrophic factor: role in depression and suicide. Neuropsychiatric Disease and Treatment 2009: 5 433-449.
3. Zanos P, Moaddel R, Morris P J, Georgiou P, Fischell J, Elmer G I et al. NMDAR inhibition-independent antidepressant actions of ketamine metabolites. Nature 2016; 533: 481-486
4. Zarate C A Jr, Singh J B, Carlson P J, Brutsche N E, Ameli R, Luckenbaugh D A, Charney D S, Manji H K. A randomized trial of an N-methyl-D-aspartate antagonist in treatment-resistant major depression. Arch Gen Psychiatry. 2006 August; 63(8):856-64.
5. Wang C, Zhou Y, Zheng W, Liu W, Zhan Y, Li H, Chen L, Zhang B, Walter M, Li M, Li M D, Ning Y. Association between depression subtypes and response to repeated-dose intravenous ketamine. Acta Psychiatr Scand. 2019 November; 140(5):446-457.
6. Bahji A, Vazquez G H, Zarate C A Jr. Comparative efficacy of racemic ketamine and esketamine for depression: A systematic review and meta-analysis. Affect Disord 2021 Jan. 1; 278:542-555

### Example 3—Norketamine Concentrations Following Wafermine (Sublingual Ketamine) Single Dose Administration

In this study, a population pharmacokinetic (PK) model was used to simulate ketamine and norketamine plasma concentrations after Wafermine single dose (sublingual) administration. The data for the population PK model development originated from plasma concentrations of ketamine and nor ketamine from study KET010 (86 patients on active treatment, with sparse sampling) and study KET012 (12 healthy subjects on active treatment, with frequent sampling). A brief summary of those studies is now provided.

Study KET010 was a phase 2, multiple dose study of the efficacy and safety of Wafermine in acute post-operative pain following bunionectomy or abdominoplasty.

The study participants who underwent bunionectomy (n=85) were randomized (1:1:1) to:
Treatment A: Wafermine 50 mg
Treatment B: Wafermine 75 mg
Treatment C: Placebo
   and the study participants who underwent abdominoplasty (n=40) were randomized (1:1:1:1) to:
Treatment A: Wafermine 50 mg
Treatment B: Wafermine 75 mg
Treatment C: Placebo
Treatment D: Wafermine 25 mg

Wafermine was administered as needed for a total of 12 hours post initial dose. The protocol employed both a fixed and flexible dosing regimen. The fixed component of the regimen required subjects to receive a dose of the study medication at least every two hours from the last dose given. The flexible portion of the regimen allowed the Investigator to recommend administration of the study medication earlier than the fixed two-hour time point. For doses #2-#5, the Investigator could recommend administration of the study medication as frequently as every 30 minutes. Subsequent doses (dose #6 and onwards) could be given as frequently as every hour.

Blood samples for PK assessment of total ketamine and norketamine were collected pre-dose, just before the 2nd, 3rd and 4th dose, just before the last dose (defined as the first dose given from 10 hours post-initial dose) and 3-8 hours after the last dose. A PK sample was also been drawn at time of early termination, if prior to 12 hours.

Study KET020 was a phase 1, randomised, open-label, three-way crossover, PK study of a single dose of two formulations of Wafermine and Ketalar in healthy subjects under fasted conditions.

The healthy subjects were randomly assigned to a treatment sequence for study drug administration:
Treatment A: Wafermine 25 mg, formulation A, sublingual
Treatment B: Wafermine 25 mg, formulation B, sublingual
Treatment C: Ketamine (US Ketalar®, 10 mg, intravenous infusion

Each treatment was separated by a washout period of a minimum of 3 days.

A total of 17 blood samples for PK assessment of total ketamine and norketamine were collected per participant per occasion (pre-dose and at 5, 10, 20, 30, 40, 50 minutes and 1, 1.25, 1.5, 2, 3, 4, 6, 10, 14 and 24 hours post dose).

### Simulated Ketamine and Norketamine Plasma Concentrations

For each scenario, 500 patients were simulated. Simulated body weights were uniformly distributed between 50 and 110 kg. Simulated single doses were: 50 mg, 75 mg, 100 mg, 125 mg, 150 mg, 175 mg, 200 mg, 225 mg, 250 mg and 275 mg.

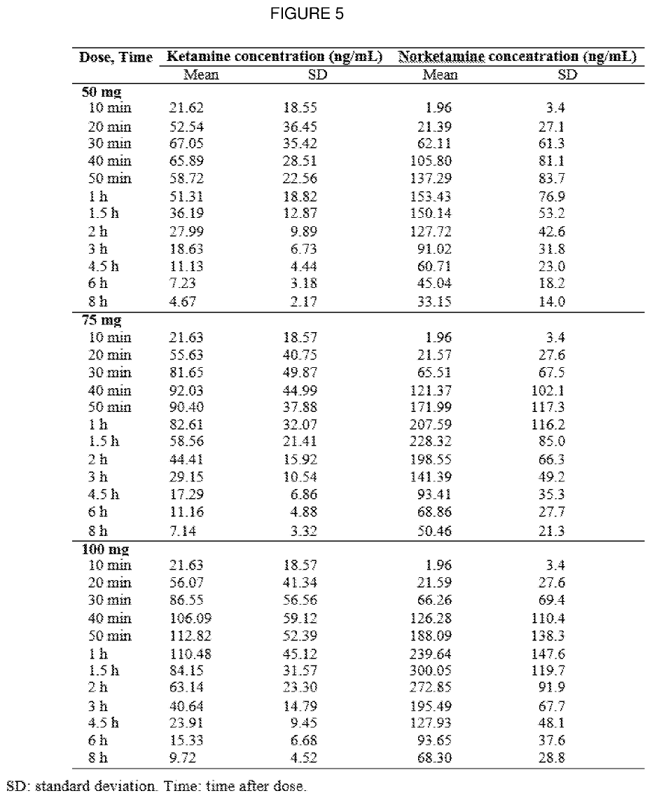

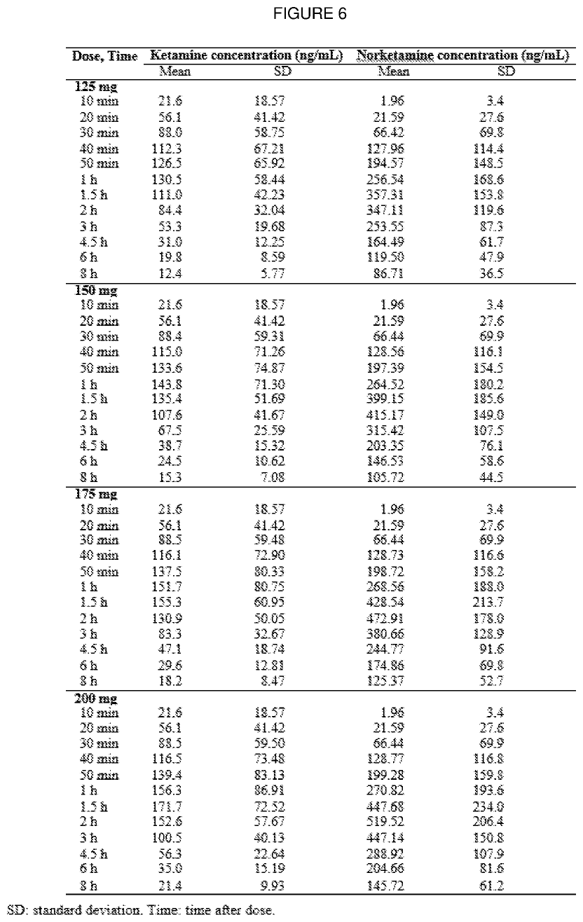

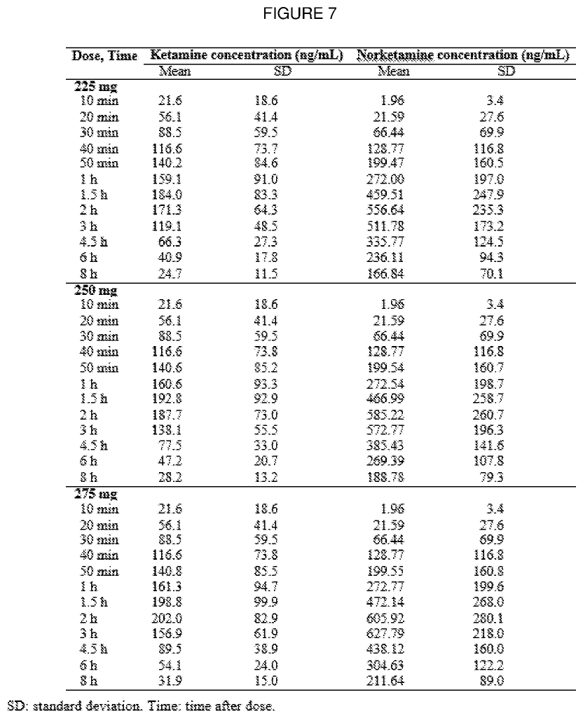

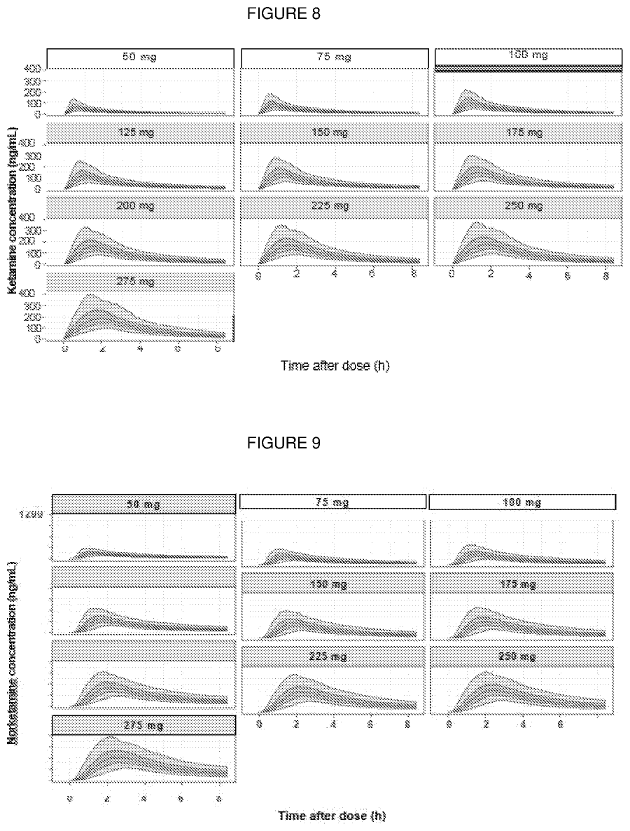

The simulated data are presented in FIGS. 5 to 9.

### Example 4—Bioavailability of Racemic Ketamine from a Novel Sublingual Formulation

### Aim

The principal study objective was to investigate the pharmacokinetic characteristics of the sublingual ketamine wafer, as defined herein, and to establish its absolute bioavailability and local tolerability.

### Methods

The study was approved by the Royal Adelaide Hospital Human Research Ethics Committee and was registered with the Australian Therapeutic Goods Administration under the Clinical Trial Notification scheme and with the Australian and New Zealand Clinical Trials Registry (Number: 2011/0292). The study was conducted in accordance with the principles of the Declaration of Helsinki and Good Clinical Practice Guidelines.

### Design

The study was of open label two way randomized, crossover design in eight healthy male volunteers who all gave written informed consent. Each participant received either a single 10 mg i.v. dose as a constant rate 30 min infusion or a 25 mg SL dose of ketamine in two treatment periods with a 7 day washout. Both the SL and i.v. doses, and the duration of the i.v. infusion were chosen to ensure adequate characterization of the plasma concentration— time profiles and good quality estimates of pharmacokinetic (PK) variables for both routes of administration. The i.v. dose of 10 mg has been used in similar studies and has been well tolerated. Bioavailability values of 24-32.2% have been reported in the literature for sublingually administered ketamine. Even if the bioavailability of the wafer formulation was higher, a 25 mg dose was not expected to show a systemic tolerability markedly different from that of the i.v. dose. The sequence of the two formulations was according to a computer-generated randomization code.

### Clinical

The SL wafer formulation was a freeze dried solid dispersion of racemic ketamine hydrochloride in a porous matrix using lactose as a filling agent. Prior to administration of the wafer the sublingual space was rinsed with 3 ml of water after which the wafer was placed sublingually by a member of the study staff. The participants were instructed to avoid chewing or swallowing of the wafer within 5 min of its placement. For i.v. administration, commercially available ketamine (Ketalar®) was diluted to 30 ml in saline and administered over 30 min using a volumetrically controlled syringe driver. The infusion line was primed prior to start of the infusion.

Pharmacokinetic blood sampling and clinical assessment of local tolerability and safety were carried out for 24 h following both dosing occasions.

Key inclusion criteria were healthy adult males aged 18-65 years with a BMI 19-30 kg m⁻² in good general health including mental health as assessed by the Symptom Checklist-90-R (SCL-90-R®), a screening instrument which evaluates a broad range of psychological problems and symptoms of psychopathology. Pharmacokinetic blood samples (5 ml), were taken following both i.v. and SL administration at predose 5, 10, 15, 30, 35 and 45 min, and at 1.0, 1.5, 2.0, 2.5, 3.0, 4.0, 6.0, 8.0, 12 and 24 h post-dose.

Whole blood was drawn into prechilled lithium heparin tubes and remained on ice post-sample collection until centrifugation. Samples were centrifuged at 1800 g for 10 min in a refrigerated centrifuge at 4° C. Plasma was decanted and frozen at −80° C.

To assess the local tolerability profile of the SL formulation, modified Likert scales (0-10) were recorded at 5, 10, 15, 30 and 45 min and 1 h post-dose administration at various time points for both the SL and i.v. formulation:
Mucosal irritation
Burning sensation
Bitterness
Nausea
Residual grittiness in the mouth
   Safety assessments included scheduled adverse event probes, spontaneous adverse event (AE) reporting, physical examination, routine laboratory investigations, ECGs and vital sign evaluation.

Vital signs (including systolic and diastolic blood pressure, pulse, respiratory rate and body temperature) were performed predose and at hours 0.5, 1, 2, 4, 6, 8, 12 and 24 h post-dose. Pulse oximetry was recorded predose and continuously for the first 3 h post-dose administration

### Laboratory

Safety laboratory testing (biochemistry, haematology and urinalysis) was performed predose and at hour 24 post-dose administration in each period. Quantification of the plasma concentrations of racemic ketamine was performed using a validated HPLC method with u.v. detection, a lower limit of quantification (LLOQ) of 2 ng ml⁻¹ and <20% bias and imprecision.

### Data Analysis

Standard non-compartmental methods using the PK Solver plug-in for Microsoft Excel were used to derive pharmacokinetic variables, except for Cₘₐₓ, Tₘₐₓ and Tₗₐₛₜ, which were taken as observations from the plasma concentration-time profile of each participant. Actual times were used when reporting Tₘₐₓ. The terminal rate constant (λ_{Z}) was estimated by log-linear regression, of the slope of the natural log plasma concentration vs. time curve where λ_{Z}=−1×slope. The linear regression in the terminal phase used the last three to six data points. The terminal t½ was calculated as t½=In(2)/λ_{Z}.

The area under the plasma concentration time curve from time zero to the last quantifiable concentration (AUC(0,tₗₐₛₜ)) was obtained using the linear trapezoidal method and extrapolated to infinity to obtain the total area, AUC(0,∞), with Cₗₐₛₜ/λ_{Z}, where Cₗₐₛₜ is the last quantifiable plasma concentration. The AUCₑₓₜᵣ (extrapolated portion of AUC(0,∞)) was calculated as (1−AUC(0,tₗₐₛₜ)/AUC(0,∞)×100. For the i.v. dose, clearance (CL) was calculated as dose/AUC(0.∞) and V_{Z} was calculated as CL/λ_{Z}. The bioavailability (F) of ketamine was calculated as the ratio of the dose adjusted AUC(0,∞) following i.v. and SL dosing according to AUC(0,∞)_{(SL)}/AUC(0,∞)_{(i.v.)}×dose_{i.v.}/dose_{SL}.

### Results

Eight healthy male volunteers of mean (SD) 25 (7.6) years and BMI 26.1 (2.83) kg m⁻² took part in the study.

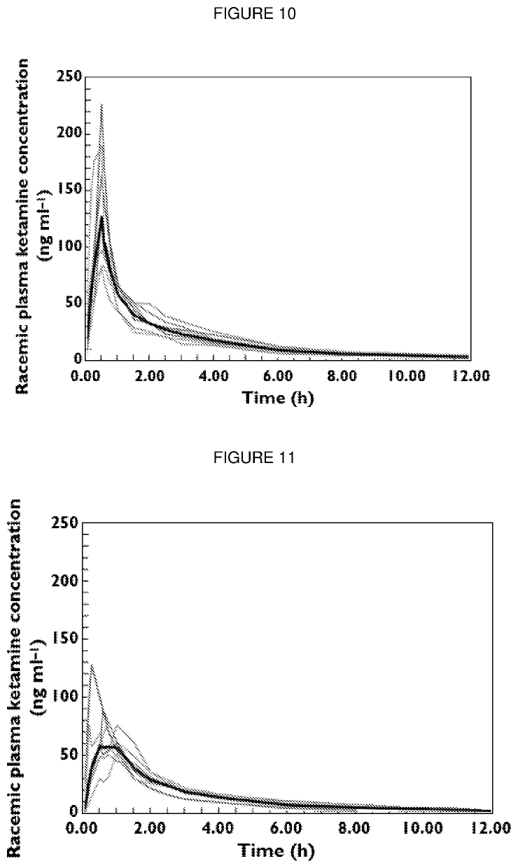

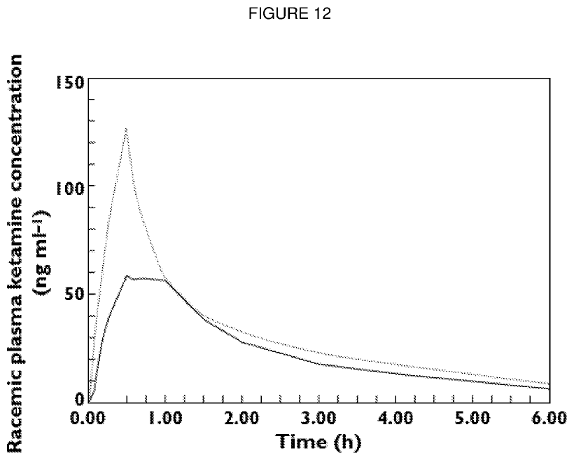

The individual and mean plasma concentration profiles are shown graphically for i.v. and SL administration in FIGS. 10 and 11, respectively. The mean profiles for i.v. and SL are shown in FIG. 12.

The pharmacokinetic results are provided in Table 2.

TABLE 2
Individual pharmacokinetic variables and summary statistics of RS ketamine following administration
of 10 mg as a 30 min i.v. infusion and 25 mg sublingually as a wafer to eight healthy volunteers.

| | [symbol] | [symbol] | t [symbol] | AUC(0,∞) [symbol] | AUC(0,∞) [symbol] | CL | V [symbol] | [symbol] | [symbol] | F |
|---|---|---|---|---|---|---|---|---|---|---|
| Subject | (ng/ml⁻¹) | (ng/ml⁻¹) | (h) | (ng ml⁻¹ h) | (ng ml⁻¹ h) | (l h⁻¹) | (l) | (h) | (h) | (%) |
| 1 | 226.7 | 88.8 | 0.58 | 282.7 | 202.9 | 35.4 | 126 | 2.5 | 2.9 | 28 |
| 2 | 166.3 | 128.3 | 0.25 | 243.2 | 162.5 | 41.1 | 158 | 2.7 | 1.8 | 27 |
| 3 | 160.3 | 76.7 | 0.75 | 254.8 | 134.3 | 39.3 | 283 | 5.0 | 3.2 | 29 |
| 4 | 124.2 | 60.2 | 1 | 270.0 | 203.5 | 37.0 | 253 | 4.7 | 5.5 | 30 |
| 5 | 120.4 | 50.0 | 0.75 | 289.2 | 171.9 | 34.6 | 300 | 6.0 | 3.5 | 23 |
| 6 | 101.9 | 75.1 | 1 | 299.4 | 211.3 | 33.4 | 164 | 3.4 | 2.3 | 29 |
| 7 | 83.2 | 51.8 | 1 | 261.0 | 166.1 | 36.3 | 385 | 7.0 | 4.6 | 29 |
| 8 | 81.1 | 61.2 | 0.5 | 167.2 | 151.6 | 59.8 | 375 | 4.3 | 5.1 | 38 |
| Gmean* | 128.1 | 71.1 | 0.25 | 255.0 | 184.6 | 39.2 | 237 | 4.5 | 3.4 | 29 |
| Min-max | 81.1-226.7 | 50.0-128.3 | 0.25-1 | 167.2-299.4 | 161.6-211.3 | 33.4-59.8 | 126-385 | 2.5-7.0 | 1.8-5.5 | 23-38 |
| CV (%) | 16 | 14 | 21 | 8 | 4 | 8 | 18 | 16 | 17 | 6 |
| 90% CI† | | | | | | | | | | 27.31 |

[symbol]

indicates data missing or illegible when filed

Cmax, peak plasma concentration; tmax, time of Cmax; AUC(0,∞), area under the plasma concentration—time curve from time zero to infinity; CL, clearance following i.v. administration; Vz, apparent volume of distribution following i.v. administration; t½, terminal half-life; F, bioavailability; NA, Not applicable; SL, sublingual. *Gmean is provided for all variables except for bioavailability, tmax and t½ where medians are shown. †90% confidence interval (lower, upper).

In all participants and for both administration routes, the first quantifiable ketamine plasma concentrations were observed at the first post-dose sample at 5 min. The SL plasma concentration profiles showed minor fluctuations in a few participants. In one participant three comparable peaks were observed during the first 1.5 h following SL administration, although no noticeable difference in PK characteristics could be observed in comparison with the other participants. Following the Cₘₐₓ, concentrations declined biphasically for both i.v. and SL with the trend being more prominent for i.v. Peak plasma concentrations following the i.v. infusion occurred at the end of the infusion in all but one participant, where the peak occurred 5 min after the end of the infusion. For the SL formulation, peak plasma concentrations were observed between 0.25 and 1 h, with a median tmax of 0.75 h. In one participant the dissolution time of the wafer was noticeably longer, 6 min, than the 30-60 s noted in all other participants. The same participant showed among the highest scores for ‘residual grittiness’ during the first 30 min after dosing, but scores had returned to 1 at 45 min and to baseline values at 60 min post-dose. The longer dissolution time did not translate into generally differing PK or systemic tolerability characteristics of ketamine in this participant. The cause of the prolonged dissolution time is unknown. Plasma concentrations were below the LLOQ in six participants at 24 h and in one participant at 12 h following SL dosing. Following i.v. dosing, all participants had quantifiable levels at 12 h and four participants at 24 h. The median (min−max) terminal half-lives for i.v. and SL were comparable at 4.5 (2.5-7.0) h and 3.4 (1.8-5.5.) h, respectively. The extrapolated portion of the AUC(0,∞) was very small for both routes of administration with min−max of 3-7% for i.v. and 2-9% for SL dosing. The median (lower, upper 90% CI limit) for the bioavailability of the wafer was 29 (27, 31) % showing very low inter-subject variability. The participant who had the highest bioavailability, 38%, also had the highest clearance, 59.8 l h⁻¹. Nineteen adverse events thought to be related to treatment were reported. Most were expected CNS-type effects typical of ketamine: light headed (n=1 for i.v. and n=3 for SL), hazy (i.v. n=2), numbness in mouth and/or face (i.v. n=5, SL n=1), and one each of body feels heavy, dry mouth and visual disturbance for i.v., and for SL one each of terrible taste in mouth, blurred vision, decreased sensation in arm and dizziness, respectively. The onset was comparable for the two routes of administration, being 6-22 min for i.v. and 5-18 min for SL dosing. All AEs were mild and had a short duration of less than 1 h with only three AEs ‘possibly’ or ‘probably’ related to treatment lasting over 30 min. There were no serious adverse events. Local tolerability of the SL formulation was excellent with transient bitterness the only effect of note.

### DISCUSSION

In this study the pharmacokinetic characteristics and absolute bioavailability of a novel SL wafer formulation of racemic ketamine were determined, and the local tolerability was assessed. A majority of the adverse events were typical CNS effects of ketamine, and were more frequently observed for the i.v. dose, which is likely due to the higher plasma concentrations achieved in comparison with the SL dose. However, all AEs were mild, resolved within 1 h and both the local and systemic tolerability was very good for both routes of administration. The extrapolated portion of the AUC(0,∞) was very small in all participants, indicating high quality in the estimates of AUC and hence bioavailability. The dissolution and subsequent absorption following SL administration was rapid, as shown by the early quantifiable plasma concentrations. The similar terminal half-lives across dosing routes confirmed that absorption was rapid and not rate limiting for the elimination. The early tₘₐₓ was also indicative of fast absorption, in the light of the similar terminal half-life values across dosing routes. The tₘₐₓ was comparable with previously reported values for SL administration of ketamine, with a median (min−max) tₘₐₓ of 0.75 h (0.25-1 h) in the present study, a median (interquartile range) of 0.5 h (0.3-0.8 h) fora lozenge and a mean (SD) of 40 (20) min for a tablet formulation. The median bioavailability at 29% was also very similar to that observed for the lozenge formulation, median of 24% and tablet, mean of 32.2%. However what differed markedly with the novel wafer formulation compared with formulations presented in previous studies was that the between subject variability in bioavailability was noticeably lower. The 90% CI was over a very narrow range of 27-31%, in comparison with an interquartile range of 19-49% for the lozenge and a standard deviation of 8.2% for the SL tablet. It should be noted that the variability estimates for all three formulations have been derived from a small number of subjects with three healthy volunteers for the SL tablet, 10 patients for the lozenge and eight volunteers in the present trial. The low inter-subject variability in bioavailability of the novel wafer might be due to the formulation delivering a more controlled release of drug into the sublingual space than a SL lozenge or tablet. The inter-variability estimate for the novel wafer formulation will require confirmation in future trials in a larger number of subjects. In the context of a narrow therapeutic index drug such as ketamine, reliable and consistent delivery is particularly important and hence the low variability in bioavailability makes the new wafer formulation especially attractive for further evaluation as an analgesic adjunct.

In summary, the clinical safety and tolerability of ketamine and the adverse event profile was as expected for the dose levels used and prevailing clinical experience and mild and transient local effects were seen. The bioavailability of ketamine in the novel SL wafer formulation was comparable with previously reported SL formulations and in addition promises a very low inter-subject variability. In view of ketamine's relatively narrow therapeutic index, low variability is appealing as it signifies reproducible exposure and consequently clinical effect.

### CONCLUSION

Sublingual administration of the ketamine wafer resulted in rapid absorption. The ketamine wafer has comparable bioavailability with other oral transmucosal formulations of ketamine but with markedly reduced inter-subject variability, warranting further evaluation as an analgesic adjunct

1. A method of treating depression comprising administering to a patient in need thereof, a fast dissolving wafer solid dosage form with a matrix for release of a biologically active material in an oral cavity wherein said dosage form comprises:
(a) a biologically active material;
(b) a matrix forming agent;
wherein the dosage form dissolves in the oral cavity without leaving a residue of said
dosage form in the oral cavity that is detectable by a subject, thereby avoiding the urge for the subject to swallow the dosage form; and
wherein said dosage form disintegrates in the oral cavity in a time of less than 15 seconds and dissolves in the oral cavity in a time of less than 60 seconds.

2. A method of claim 1, wherein the solid dosage form is delivered sublingually.

3. A method according to anyone of the above claims, wherein wherein the wafer is freeze-dried.

4. A method according to anyone of the above claims, wherein wherein the solid dosage form is fast disintegrating.

5. A method according to anyone of the above claims, wherein wherein the biologically active material is absorbed by diffusion directly into the systemic circulation.

6. A method according to anyone of the above claims, wherein the biologically active material is selected from the group consisting of: ketamine, an analog, variant, metabolite and a salt form thereof.

7. A method according to anyone of the above claims, wherein the ketamine is present in an amorphous (non-crystalline) state.

8. A method according to anyone of the above claims, wherein the matrix forming agent comprises amylopectin.

9. A method according to anyone of the above claims, wherein the amylopectin is not in the form of a starch or modified starch.

10. A method according to anyone of the above claims, wherein the matrix forming agent comprises a carbohydrate.

11. A method according to anyone of the above claims, wherein the powder x-ray diffraction (XRD) spectrum of the dosage form comprises peaks at 2-theta values at approximately 9.58 degrees, 19.68 degrees, and 20.05 degrees.

12. A method according to anyone of the above claims, wherein the solid dosage form is porous.

13. A method according to anyone of the above claims, wherein the depression is selected from the group consisting of: major depressive disorder or treatment-resistant depression.

14. A method according to anyone of the above claims, wherein the solid dosage form comprises a dose of ketamine selected from the group consisting of: 25 mg, 50 mg, 75 mg, 100 mg, 150 mg.

15. A method according to anyone of the above claims, wherein the solid dosage form provides an effective plasma concentration of ketamine material within a period of no more than two hours, 30 minutes, 20 minutes, or 15 minutes.

16. A method according to anyone of the above claims, wherein the solid dosage form provides a Cₘₐₓ at a comparable time to an IV injection but lower than that of an injection of the same dosage.

17. A method according to anyone of the above claims, wherein the solid dosage form provides a tₘₐₓ at a comparable time to an IV injection.

18. A method according to anyone of the above claims, wherein the ketamine is rapidly absorbed with detectable concentrations at the first sampling time of 3 minutes.

19. A method according to anyone of the above claims, wherein the dosage form is administered to the subject utilising a dosing regimen selected from the group consisting of: at a frequency to alleviate the symptoms of depression, twice hourly, once every six hours, once every 12 hours, once daily, twice weekly, once weekly, once every two weeks, once a month, every two months, once every six months, once yearly.

20. A method for improving compliance with a ketamine prescription in a patient suffering depression, said method comprising the method according to anyone of the above claims.
